(19) United States
(12) Patent Application Publication
(10) Pub. No.: US 2025/0160265 A1
(43) Pub. Date: May 22, 2025

(54) SYSTEM FOR MONITORING GREENHOUSE GASES IN FIELD

(71) Applicant: National Pingtung University of Science and Technology, Neipu Township (TW)

(72) Inventors: Chin-Lung CHANG, Neipu Township (TW); Cheng-Huei YANG, Tainan City (TW); Yu-Min WANG, Pingtung City (TW)

(21) Appl. No.: 18/953,793

(22) Filed: Nov. 20, 2024

(30) Foreign Application Priority Data
Nov. 21, 2023 (TW) 112144938

(51) Int. Cl. A01G 9/14; A01G 9/24

(52) U.S. Cl. CPC A01G 9/14; A01G 9/247

(57) ABSTRACT

A system for monitoring greenhouse gases in a field has a host server that performs data processing and calculations, and that stores a plurality of management datasets. The system includes a monitoring unit disposed in the field and an irrigation control unit. The monitoring unit includes a wireless communication module that receives and transmit data, a greenhouse gas monitoring module that detects greenhouse gas data, a sensing module that generates soil data, distance data, temperature data and humidity data related to the field, and a controller that receives and transmits the greenhouse gas data, the soil data, the distance data, the temperature data and the humidity data to the host server through the wireless communication module, so that the controller receives control data from the host server. The irrigation control unit adjusts a water level of the field based on the control data received from the monitoring unit.

### CROSS-REFERENCE TO RELATED APPLICATION

This application claims priority to Taiwanese Invention Patent Application No. 112144938, filed on Nov. 21, 2023, the entire disclosure of which is incorporated by reference herein.

### FIELD

The disclosure relates to an agricultural monitoring system, and more particularly to a system for monitoring greenhouse gases in a field.

### BACKGROUND

Agricultural development is closely tied to a country's food supply. Furthermore, when there is a surplus of food supply, surplus food can be exported to contribute to the country's economy. However, in recent years, extreme weather (e.g., heat waves, droughts, forest fires, floods, blizzards, etc.) has not only occurred more frequently, but has also become increasingly unpredictable. Global warming, driven mainly by greenhouse gas emissions, is one of the primary causes of extreme weather. In terms of the relationship between greenhouse gases and agricultural development, although some crops may absorb carbon dioxide during photosynthesis, agricultural activities may contribute to the emission of other greenhouse gases such as methane, ozone, nitrous oxide, etc. For example, in rice cultivation, the soil may release methane from soil conditioning, fertilizer application, etc. Additionally, synthetic fertilizers, livestock manure, nitrogen-fixing crops, and crop residues may release nitrous oxide. Furthermore, agricultural infrastructures, such as irrigation systems, farm machinery, and electrical equipment, may contribute to carbon emissions.

Therefore, when considering the long-term operation and sustainable development of agriculture, even though carbon emission cannot be completely prevented, it is important to adopt better agricultural practices in order to achieve a reasonable balance between food production and sustainable development.

### SUMMARY

Therefore, an object of the disclosure is to provide a system for monitoring greenhouse gases in a field that can alleviate at least one of the drawbacks of the prior art.

According to the disclosure, a system for monitoring greenhouse gases in a field is adapted to communicate with a host server that is configured to perform data processing and calculations and that stores a plurality of management datasets. The system includes a monitoring unit that is adapted to be disposed in the field and that is configured to communicate with the host server, and an irrigation control module. The monitoring unit includes a wireless communication module that is configured to receive and transmit data, a greenhouse gas monitoring module, a sensing module, and a controller. The greenhouse gas monitoring module has a container that surrounds and defines an inner space, a gas sensor that is disposed in the inner space and that is configured to detect greenhouse gas data, an actuator that is adapted to allow the gas sensor to be disposed thereon and that is configured to be actuated so as to adjust a position of the gas sensor in the inner space, a connecting pipe that is configured to allow spatial communication between the inner space and a space outside the container, an air valve that is disposed in the connecting pipe, and a fan that is disposed in the connecting pipe and that is configured to control flow of gas into and out of the connecting pipe. The sensing module is configured to obtain a plurality of parameters related to the field, and includes a soil sensor that is configured to generate soil data, a distance sensor that is configured to generate distance data, and a weather sensor that is configured to generate temperature data and humidity data. The controller is electrically connected to the wireless communication module, the greenhouse gas monitoring module and the sensing module, and is configured to receive the greenhouse gas data from the greenhouse gas monitoring module, to receive the soil data, the distance data, the temperature data and the humidity data from the sensing module, and to transmit, through the wireless communication module, the greenhouse gas data, the soil data, the distance data, the temperature data and the humidity data to the host server, so that the controller receives control data from the host server, where the control data is generated by the host server based on the greenhouse gas data, the soil data, the distance data, the temperature data and the humidity data received from the controller, and the management datasets stored in the host server. The irrigation control unit is electrically connected to the monitoring unit, and is configured to adjust a water level of the field based on the control data received from the monitoring unit.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a schematic view illustrating a system for monitoring greenhouse gases in a field according to an embodiment of the disclosure.

FIG. 2 is a block diagram illustrating a monitoring unit of the system according to an embodiment of the disclosure.

FIG. 3 is a schematic view illustrating a controller, a greenhouse gas monitoring module and a sensing module of the monitoring unit according to an embodiment of the disclosure.

FIG. 4 is a block diagram illustrating an irrigation control unit of the system according to an embodiment of the disclosure.

FIG. 5 is a schematic view illustrating an application program installed in a remote unit of the system according to an embodiment of the disclosure.

FIGS. 6 to 9 are schematic views illustrating functions of the application program according to an embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

Throughout the disclosure, the term “connected to” may refer to a direct connection among a plurality of electrical apparatus/devices/equipment via an electrically conductive material (e.g., an electrical wire), or an indirect connection between two electrical apparatus/devices/equipment via another one or more apparatus/devices/equipment, or wireless communication.

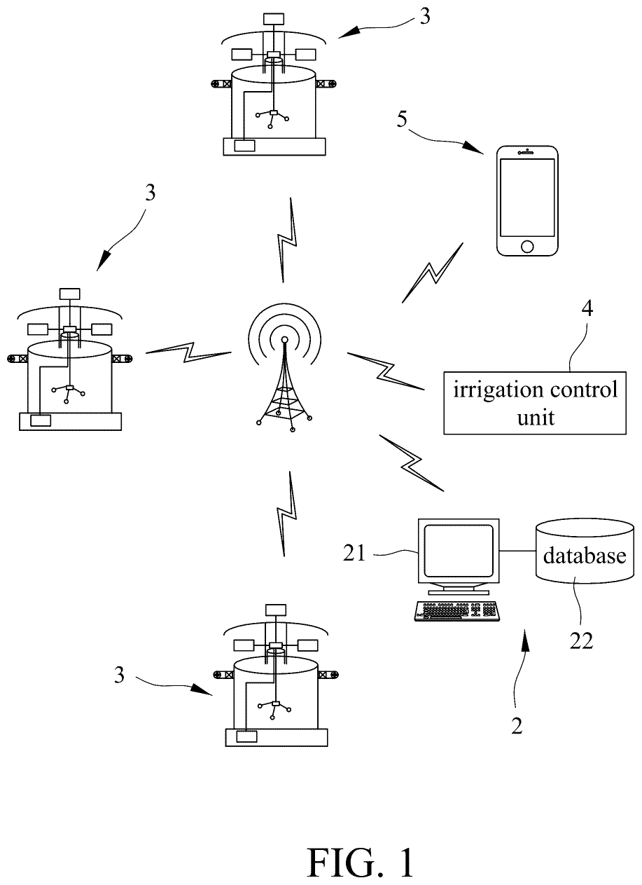

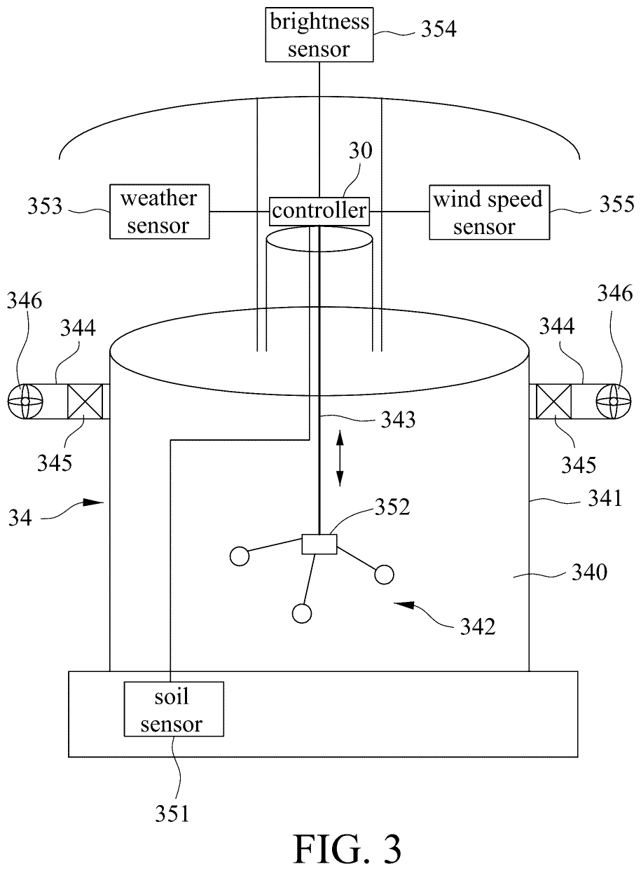

Referring to FIGS. 1 to 3, according to an embodiment of the disclosure, a system for monitoring greenhouse gases in a field includes a host server 2, a plurality of monitoring units 3 that are adapted to be disposed in at least one field and that are electrically connected to the host server 2, an irrigation control unit 4 that is electrically connected to the monitoring units 3, and a remote unit 5 that is electrically connected to the monitoring units 3 and the irrigation control unit 4. It should be noted that a quantity of the monitoring units 3 disposed in each field may depend on a size or a condition of the field. In one example, for a large field, multiple monitoring units 3 may be disposed in the large field, and for a multiple of small fields, one monitoring unit 3 may be disposed in each of the small fields.

The host server 2 includes a processor 21 that is configured to perform data processing and calculations, and a database 22 that is electrically connected to the processor 21 and that stores a plurality of management datasets. In this embodiment, the host server 2 is a terminal server that uses the processor 21, which has powerful computing capabilities, to deal with large computing demands of the system, and the database 22 provides the management datasets to the processor 21 for the processor 21 to perform necessary computations. Specifically, the management datasets may include data related to a specific crop (e.g., greenhouse gas emission data, environmental parameters or irrigation requirements of the specific crop), or may include empirical data collected over the years. As such, the management datasets may provide a reference for a user to make plans or to control the system for growing crops in the field, or may be provided to the processor 21 for the processor 21 to perform integrated planning for growing crops in the field.

Since each of the monitoring units 3 in the disclosure operates in the same manner, only one monitoring unit 3 will be described in detail in the following description. The monitoring unit 3 includes a controller 30, and an operating interface 31, a positioning system 32, a wireless communication module 33, a greenhouse gas monitoring module 34 and a sensing module 35 that are electrically connected to the controller 30. The positioning system 32 is configured to obtain position data related to a position of the monitoring unit 3. The wireless communication module 33 is configured to receive and transmit data. The sensing module 35 is configured to obtain a plurality of parameters related to the field where the monitoring unit 3 is disposed.

In this embodiment, the controller 30 may be implemented by a central processing unit (CPU), a microcontroller (MCU), or any processing chip with calculation and processing functions, and may be installed with specific programs in advance so that the controller 30 is capable of performing controls corresponding to various logic conditions, thus becoming the core controller of the system. In this embodiment, the operating interface 31 may include, for example, dual in-line package (DIP) switches, buttons, or a touch interface for the user to operate for controlling the greenhouse gas monitoring module 34 and the sensing module 35, but the disclosure is not limited to such. In this embodiment, the positioning system 32 may be a global positioning system (GPS). The wireless communication module 33 may include one or more of a short-range wireless communication module supporting a short-range wireless communication network using a wireless technology such as Bluetooth® and/or Wi-Fi, etc., and a mobile communication module supporting telecommunication using Long-Term Evolution (LTE), the third generation (3G), the fourth generation (4G) or fifth generation (5G) of the wireless mobile telecommunications technology, or the like.

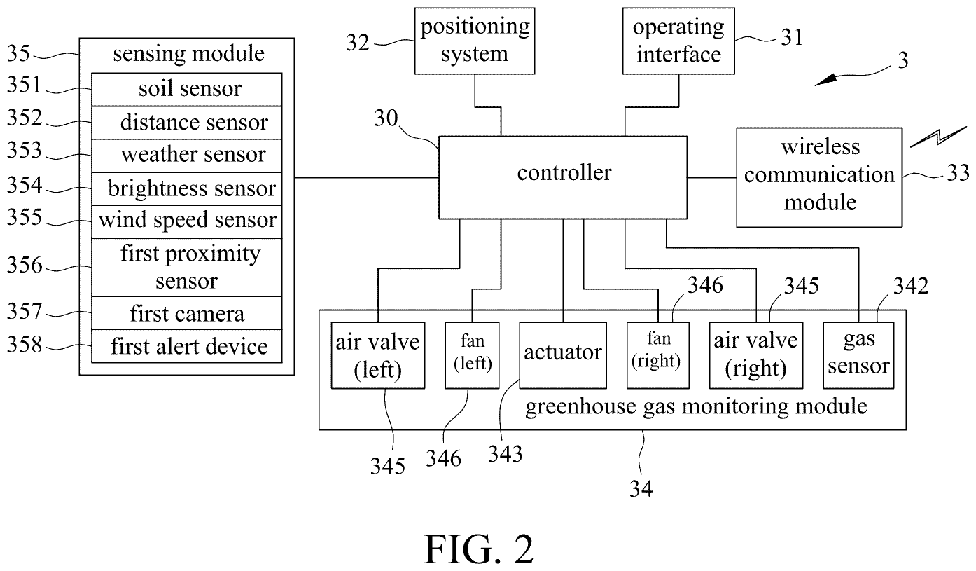

The greenhouse gas monitoring module 34 includes a container 341 that surrounds and defines an inner space 340, a gas sensor 342 that is disposed in the inner space 340 and that is configured to detect greenhouse gas data, an actuator 343 that is adapted to allow the gas sensor 342 to be disposed thereon and that is configured to be actuated so as to adjust a position of the gas sensor 342 in the inner space 340, two connecting pipes 344 that are configured to allow spatial communication between the inner space 340 and a space outside the container 341, two air valves 345 (represented as air valve (left) and air valve (right) in FIG. 2) that are disposed respectively in the connecting pipes 344, and two fans 346 (represented as fan (left) and fan (right) in FIG. 2) that are disposed respectively in the connecting pipes 344 and that are configured to control flow of gases into and out of the connecting pipes 344 in cooperation with the opening and the closing of the air valves 345.

Specifically, the gas sensor 342 is configured to, after gases from outside the container 341 have entered the inner space 340, obtain the greenhouse gas data related to greenhouse gases that may be produced when growing crops in the field. In one example, the greenhouse gas data may include concentrations of carbon dioxide, methane, and nitrous oxide, respectively. The actuator 343 may be automatically actuated by the controller 30, or may be manually actuated by the user (e.g., through the operating interface 31), so as to move the gas sensor 342 to an advantageous position for measuring the greenhouse gas data (e.g., to a specific height relative to the ground surface or relative to the water surface of the field) according to user needs. For example, the actuator 343 includes a motor (not shown) electrically connected to and controlled by the controller 30, and a rope connected to and driven by the motor to move vertically, where the gas sensor 342 is disposed on one end of the rope so as to move vertically along with the rope in the inner space 340. To take new measurements for the greenhouse gas data, the air valves 345 may be opened and the fans 346 may be turned on (e.g., automatically by the controller 30, or manually by the user through the operating interface 31) so as to clear out the gases inside the inner space 340, and after a while, the fans 346 may be controlled such that gases outside the inner space 340 are sucked into the inner space 340 for new measurements to be taken.

The sensing module 35 is configured to obtain a plurality of parameters related to the field, and includes a soil sensor 351 that is configured to detect soil moisture of the field so as to generate soil data, a distance sensor 352 that is configured to detect a distance to a ground level or to a water level of the field so as to generate distance data, a weather sensor 353 that is configured to detect a temperature and a humidity on the field so as to generate temperature data and humidity data, respectively, a brightness sensor 354 that is configured to detect brightness of sunlight so as to generate brightness data, and a wind speed sensor 355 that is configured to detect a wind speed on the field so as to generate wind speed data. In one embodiment, the distance sensor 352 is disposed on the gas sensor 342, where the distance data is related to a distance from the gas sensor 342 to the ground level or to the water level of the field, and the controller actuates the actuator 343 for adjusting the position of the gas sensor 342 based on the distance data.

The sensing module 35 further includes a first proximity sensor 356 that is electrically connected to the controller 30 and that is configured to detect an object approaching the first proximity sensor 356, a first camera 357 that is electrically connected to the controller 30 and that is configured to capture an image (or a video) of the field, and a first alert device 358 that is electrically connected to the controller 30 and that is configured to output a first alert signal in response to the controller 30 determining that the first proximity sensor 356 detects an object approaching. To describe in further detail, the first proximity sensor 356 and the first alert device 358 operate cooperatively so that the first alert signal is outputted to warn and drive away any stranger who wanders into the field and approaches the monitoring unit 3 within a certain distance.

It should be noted that the controller 30 receives the greenhouse gas data from the greenhouse gas monitoring module 34, and receives the soil data, the distance data, the temperature data, the humidity data, the brightness data and the wind speed data from the sensing module 35, and transmits the abovementioned data to the processor 21 through the wireless communication module 33. That is to say, the controller 30 is able to monitor and provide the environmental conditions (e.g., the abovementioned data) related to the field to the host server 2, so that the processor 21 generates, based on the environmental conditions and the management datasets (e.g., including basic growing needs of the crops), control data for controlling the monitoring unit 3 and/or the irrigation control unit 4 when any of the environmental conditions becomes abnormal, or when new measurements are required. The processor 21 then sends the control data to the controller 30 for the controller 30 to automatically control the monitoring unit 3, or for the controller 30 to further send the control data to the irrigation control unit 4 for controlling the irrigation control unit 4 (which will be described later in the description).

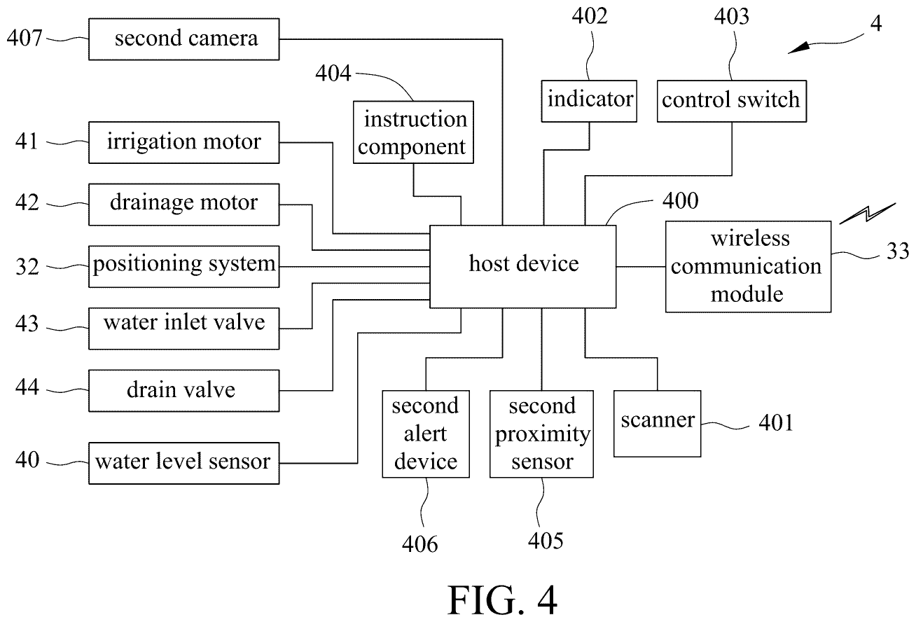

Referring further to FIG. 4, the irrigation control unit 4 includes a host device 400 (e.g., an MCU) that is configured to communicate with the controller 30 through the wireless communication module 33, a water level sensor that is electrically connected to the host device 400 and that is configured to detect the water level of the field so as to generate water level data, an irrigation motor 41 (e.g., a pumping motor) that is connected to a water source and that is configured to provide a pumping power for importing irrigation water from the water source to the field, a drainage motor 42 that is configured to provide a draining power for draining the irrigation water from the field, a water inlet valve 43 that is disposed in an irrigation channel of the field, and a drain valve 44 that is disposed downstream of the field. It should be noted that the host device 400 receives the water level data from the water level sensor 40, and provides the water level data to the controller 30 so that the controller 30 may further provide the water level data to the processor 21 for generating the control data. In one example, the irrigation control unit 4 is configured to adjust the water level of the field based on the distance data, the water level data and the control data (generated by the processor 21 and received from the controller 30 through the wireless communication module 33). Specifically, since the distance sensor 352 is able to obtain the distance data related to the distance to the ground level or to the distance to the water level of the field, the processor 21 of the host server 2 may determine the water level of the field more accurately based on both the distance data and the water level data. Additionally, the soil data generated by the soil sensor 351 may also be provided to the controller 30, so that the controller 30 may send the soil data to the host server 2 for the generation of the control data.

In one example, if the water level of the field is determined to be too low (e.g., less than 3 cm) based on the water level data and/or the distance data, or if the soil moisture is determined to be insufficient (e.g., less than 30%) based on the soil data, the control data may be generated and sent to the controller 30 of the monitoring unit 3, and the controller 30 may send the control data to the host device 400 so that the host device 400 automatically controls, based on the control data, the irrigation motor 41 to turn on and the water inlet valve 43 to open, causing the irrigation water to be imported from the water source to the field. In another example, if the water level of the field is determined to be too high (e.g., more than 5 cm), or if the soil moisture is determined to be high (which is different depending on the type of the crop), the control data may be generated so that the host device 400 automatically controls, based on the control data, the drainage motor 42 to turn on and the drain valve 44 to open, causing excessive water in the field to be drained out through the drain valve 44. It should be noted that, the thresholds for determining whether the water level and/or the soil moisture is too low or too high depends on the type of the crop that is growing in the field, and should not be limited to the abovementioned examples.

The irrigation control unit 4 further includes a scanner 401 that is electrically connected to the host device 400 and that is configured to scan an identification code (e.g., printed on an identification card) and to transmit the identification code to the host device 400 for the host device 400 to identify the identification code, an indicator 402 that is electrically connected to the host device 400 and that is configured to be controlled by the host device 400 to generate a success signal in response to the host device 400 successfully identifying the identification code, and a second alert device 406 electrically connected to the host device 400. To describe in further detail, the host device 400 is configured to identify whether the identification code received from the scanner 401 belongs to any known person of the field. When the host device 400 successfully identifies the identification code (i.e., the identification code belongs to a known person of the field), the indicator 402 generates the success signal (e.g., a light-emitting diode (LED) lights up) to indicate that the identification code has been successfully identified, and the person identified with the identification code may be allowed to operate the irrigation control unit 4. Otherwise, when the identification code fails to be successfully identified by the host device 400, the host device 400 controls the second alert device 406 to emit a second alert signal.

The irrigation control unit 4 further includes a control switch 403 that is electrically connected to the host device 400 and that is configured to be operable by the user (e.g., for controlling the irrigation motor 41, the drainage motor 42, the water inlet valve 43 and the drain valve 44) upon the host device 400 successfully identifying the identification code, and an instruction component 404 that is electrically connected to the host device 400 and that is configured to provide a set of instructions to the user for controlling the control switch 403. In one example, the set of instructions may include how to operate the control switch 403 to control the irrigation motor 41, the drainage motor 42, the water inlet valve 43 and/or the drain valve 44. As such, when the user is to manually control the irrigation control unit 4, the user is able to read the instructions from the instruction component 404 to control the control switch 403 for manually controlling the irrigation control unit 4. For example, the instruction component 404 is a lighting device (e.g., an LED light) for outputting different types of light as the instructions, or a display for displaying the instructions in a form of text, image, video, etc.

The irrigation control unit 4 further includes a second proximity sensor 405 that is electrically connected to the host device 400 and that is configured to detect an object approaching the second proximity sensor 405, and a second camera 407 that is configured to monitor operations of the irrigation motor 41 and the drainage motor 42. The second alert device 406 is configured to be controlled by the host device 400 to emit the second alert signal in response to the host device 400 determining that the second proximity sensor 405 detects an object approaching.

In this embodiment, the first camera 357 and the second camera 407 may each be an infrared camera that is capable of capturing images/videos in dark environments (e.g., during night time). In this embodiment, the first alert device 358 and the second alert device 406 may each be a display, a lighting device, an audio device, or a combination thereof, that is configured to output the first alert signal and the second alert signal, respectively, in the form of a message, light, sound or a combination thereof.

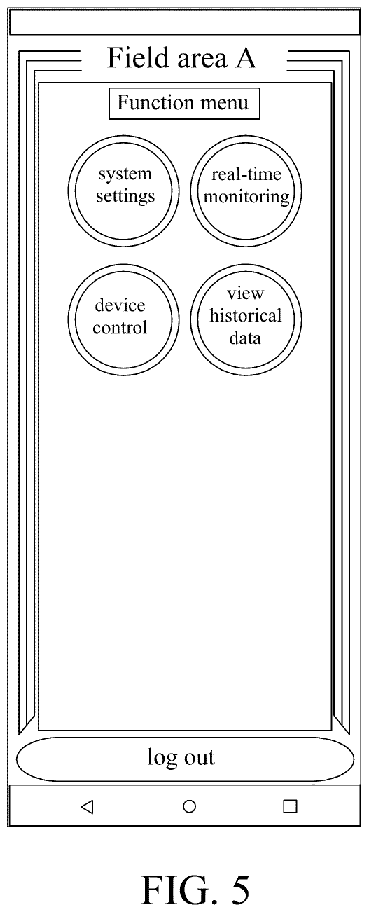

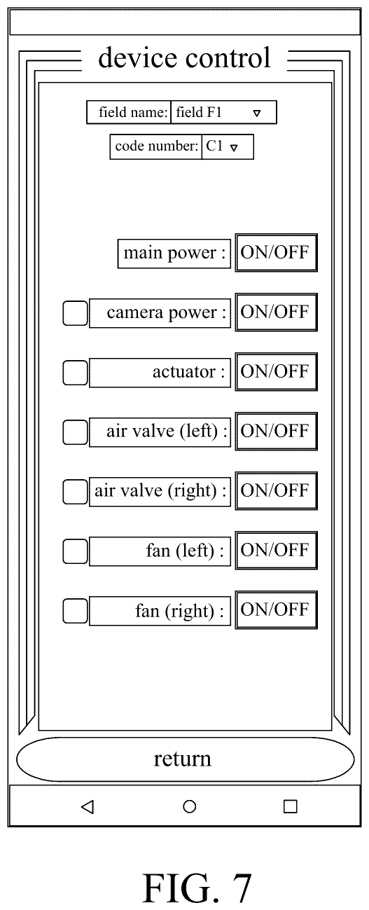

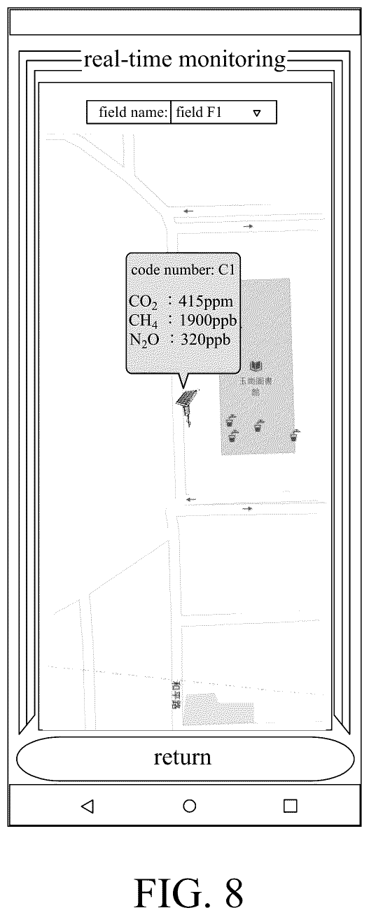

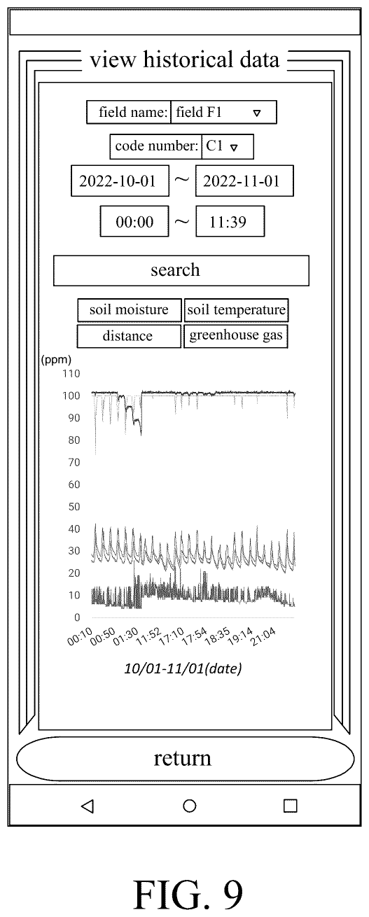

Referring further to FIGS. 5 to 9, the remote unit 5 is a mobile device (e.g., a smartphone) that has an application program installed thereon, and that is configured to receive the control data from the wireless communication module 33 of the monitoring unit 3 for controlling the monitoring unit 3 and the irrigation control unit 4. It should be noted that the controller 30 is further configured to transmit the position data obtained by the positioning system 32 to the remote unit 5 through the wireless communication module 33, and the remote unit 5 is further configured to display the position of the monitoring unit 3 based on the position data thus received. In some embodiments, the controller 30 transmits the position data obtained by the positioning system 32 to the host device 400, and the host device 400 may transmit the position data to the remote unit 5 through the wireless communication module 33.

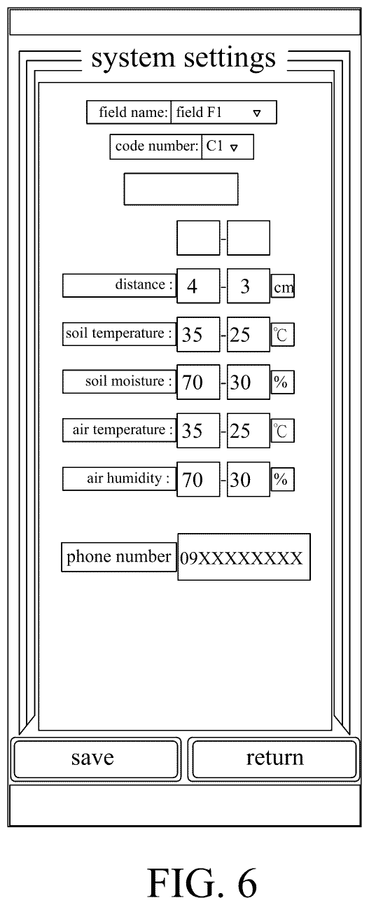

It should be further noted that when the control data is sent to the remote unit 5, the control data is displayed in a form of text for the user to read, so that the user may decide how to control operation of the monitoring unit 3 and/or the irrigation control unit 4 based on the control data (i.e., based on the text). The application program is configured to provide functions including system settings, device control, real-time monitoring, and view historical data for the monitoring unit 3 and the irrigation control unit 4 (as shown in FIGS. 6 to 9). That is to say, once the application program is installed on the remote unit 5, the remote unit 5 is able to remotely set, control and monitor the monitoring unit 3 and the irrigation control unit 4 (e.g., control operations of the irrigation motor 41, the drainage motor 42, the water inlet valve 43 and the drain valve 44 for adjusting the water level), and to view historical data (e.g., the soil data, the temperature data, the humidity data, the brightness data, the wind speed data, the water level data, the distance data, the greenhouse gas data, the position data) obtained by the monitoring unit 3 and the irrigation control unit 4. As such, the user may monitor and timely control the monitoring unit 3 and the irrigation control unit 4 when anything abnormal occurs.

In summary, according to the disclosure, the monitoring unit 3 is configured to monitor the environmental conditions of the field, and to send data related to the environmental conditions to the host server 2, so that the processor 21 of the host server 2 may generate the control data for the monitoring unit 3 and/or the irrigation control unit 4 to automatically control operation of the monitoring unit 3 and/or the irrigation control unit 4, or for the user to manually control the monitoring unit 3 and/or the irrigation control unit 4 through the remote unit 5. Moreover, the monitoring units 3 may be installed in multiple fields so as to monitor the fields simultaneously, and data related to the fields may be obtained by the host server 2 and be displayed on the remote unit 5 for the user to review, thus improving management efficiency of the fields. As such, the irrigation control unit 4 may be controlled using better agricultural practices (e.g., by controlling the water level of the field) aiming for reduction in greenhouse gas emissions from growing crops in the field.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A system for monitoring greenhouse gases in a field, the system being adapted to communicate with a host server that is configured to perform data processing and calculations and that stores a plurality of management datasets, the system comprising:
a monitoring unit adapted to be disposed in the field and configured to communicate with the host server, said monitoring unit including
a wireless communication module configured to receive and transmit data,
a greenhouse gas monitoring module including a container that surrounds and defines an inner space, a gas sensor that is disposed in said inner space and that is configured to detect greenhouse gas data, an actuator that is adapted to allow said gas sensor to be disposed thereon and that is configured to be actuated so as to adjust a position of said gas sensor in said inner space, a connecting pipe that is configured to allow spatial communication between said inner space and a space outside said container, an air valve that is disposed in said connecting pipe, and a fan that is disposed in said connecting pipe and that is configured to control flow of gas into and out of said connecting pipe,
a sensing module configured to obtain a plurality of parameters related to the field, and including a soil sensor that is configured to generate soil data, a distance sensor that is configured to generate distance data, and a weather sensor that is configured to generate temperature data and humidity data, and
a controller electrically connected to said wireless communication module, said greenhouse gas monitoring module and said sensing module, and configured to receive the greenhouse gas data from said greenhouse gas monitoring module, to receive the soil data, the distance data, the temperature data and the humidity data from said sensing module, and to transmit, through said wireless communication module, the greenhouse gas data, the soil data, the distance data, the temperature data and the humidity data to the host server, so that said controller receives control data from the host server, where the control data is generated by the host server based on the greenhouse gas data, the soil data, the distance data, the temperature data and the humidity data received from said controller, and the management datasets stored in the host server; and
an irrigation control unit electrically connected to said monitoring unit, and configured to adjust a water level of the field based on the control data received from said monitoring unit.

2. The system as claimed in claim 1, wherein said sensing module of said monitoring unit further includes a brightness sensor that is configured to generate brightness data, and a wind speed sensor that is configured to generate wind speed data.

3. The system as claimed in claim 1, wherein said irrigation control unit includes an irrigation motor that is adapted to be connected to a water source and that is configured to provide a pumping power for importing irrigation water from the water source to the field, and a drainage motor that is configured to provide a draining power for draining the irrigation water from the field.

4. The system as claimed in claim 3, wherein said irrigation control unit further includes a water inlet valve that is adapted to be disposed in an irrigation channel of the field, and a drain valve that is adapted to be disposed downstream of the field.

5. The system as claimed in claim 4, wherein said irrigation control unit further includes a host device that is configured to communicate with said controller, a scanner that is electrically connected to said host device and that is configured to scan an identification code and to transmit the identification code to said host device for said host device to identify the identification code, an indicator that is electrically connected to said host device and that is configured to be controlled by said host device to generate a success signal in response to said host device successfully identifying the identification code, a control switch that is electrically connected to said host device and that is configured to be operable upon said host device successfully identifying the identification code, and an instruction component that is electrically connected to said host device and that is configured to provide a set of instructions for controlling said control switch.

6. The system as claimed in claim 5, wherein said irrigation control unit further includes a water level sensor that is electrically connected to said host device and that is configured to generate water level data, a proximity sensor that is electrically connected to said host device and that is configured to detect an object approaching said proximity sensor, an alert device that is electrically connected to said host device and that is configured to be controlled by said host device to emit an alert signal in response to said host device determining that said proximity sensor detects an object approaching, and a camera that is configured to monitor operations of said irrigation motor and said drainage motor.

7. The system as claimed in claim 1, wherein said sensing module of said monitoring unit further includes a proximity sensor that is electrically connected to said controller and that is configured to detect an object approaching said proximity sensor, a camera that is electrically connected to said controller and that is configured to capture an image of the field, and an alert device that is electrically connected to said controller and that is configured to output an alert signal in response to said controller determining that said proximity sensor detects an object approaching.

8. The system as claim in claim 1, the system further comprising a remote unit electrically connected to said monitoring unit and said irrigation control unit, and configured to receive the control data from said monitoring unit and to control said monitoring unit and said irrigation control unit.

9. The system as claimed in claim 8, wherein said remote unit is a mobile device that has an application program installed thereon, and the application program is configured to set, control and monitor said monitoring unit and said irrigation control unit, and to view historical data obtained by said monitoring unit and said irrigation control unit.

10. The system as claimed in claim 8, wherein said monitoring unit further includes a positioning system that is electrically connected to said controller and that is configured to obtain position data related to a position of said monitoring unit, said controller is further configured to transmit the position data to said remote unit through said wireless communication module, and said remote unit is further configured to display the position of said monitoring unit based on the position data thus received.

11. The system as claimed in claim 1, wherein said monitoring unit further includes an operating interface electrically connected to said controller, and configured to allow user operation to actuate said controller for controlling said actuator, said air valve and said fan.

12. The system as claimed in claim 1, further comprising said host server including a processor that is configured to perform data processing and calculations, and a database that is electrically connected to said processor and that stores the management datasets,
wherein said processor is configured to generate the control data based on the greenhouse gas data, the soil data, the distance data, the temperature data and the humidity data received from said controller, and the management datasets stored in the database.
